(19) United States
(12) Patent Application Publication
(10) Pub. No.: US 2011/0224447 A1
(43) Pub. Date: Sep. 15, 2011

(54) Novel Lipid Nanoparticles and Novel Components for Delivery of Nucleic Acids

(76) Inventors: Keith A. Bowman, Harleysville, PA (US); James P. Guare, JR., Quakertown, PA (US); George D. Hartman, Lansdale, PA (US); Rubina G. Parmar, Harleysville, PA (US); Chandra Vargeese, Schwenksville, PA (US); Weimin Wang, Southampton, PA (US); Ye Zhang, Lower Gwynedd, PA (US)

(21) Appl. No.: 13/059,491

(22) PCT Filed: Aug. 11, 2009
(86) PCT No.: PCT/US09/53336

§ 371 Date: Feb. 17, 2011

Related U.S. Application Data
(60) Provisional application No. 61/189,295, filed on Aug. 18, 2008.

(51) Int. Cl. C07J 9/00; A61K 31/713; A61P 1/16

(52) U.S. Cl. 552/544; 514/44 A

(57) ABSTRACT

The instant invention provides for novel lipid nanoparticles and novel lipid nanoparticle components (specifically cationic lipids) that are useful for the delivery of nucleic acids, specifically siRNA, for therapeutic purposes.

### BACKGROUND OF THE INVENTION

The present invention relates to lipid nanoparticles, lipid nanoparticle components (specifically cationic lipids) and methods for delivering biologically active molecules in vitro and in vivo. Specifically, the invention relates to lipid nanoparticles, lipid nanoparticle components (specifically cationic lipids) and methods for delivering nucleic acids, polynucleotides, and oligonucleotides such RNA, DNA and analogs thereof, peptides, polypeptides, proteins, antibodies, hormones and small molecules for therapeutic purposes. More specifically, the invention relates to lipid nanoparticles, lipid nanoparticle components (specifically cationic lipids) and methods for delivering siRNA and miRNA for therapeutic purposes.

Cationic lipids and the use of cationic lipids in lipid nanoparticles for the delivery of biologically active molecules, in particular siRNA and miRNA, has been previously disclosed. (See US patent applications: U.S. 2006/0240554 and U.S. 2008/0020058). Lipid nanoparticles and the use of lipid nanoparticles for the delivery of biologically active molecules, in particular siRNA and miRNA, has been previously disclosed. (See US patent applications: U.S. 2006/0240554 and U.S. 2008/0020058). siRNA and the synthesis of siRNA has been previously disclosed. (See US patent applications: U.S. 2006/0240554 and U.S. 2008/0020058).

It is an object of the instant invention to provide novel lipid nanoparticles and novel lipid nanoparticle components (specifically cationic lipids) that are useful for the delivery of nucleic acids, specifically siRNA, for therapeutic purposes. The lipid nanoparticles of the instant invention provide unexpected properties, in particular, enhanced efficacy, relative to other lipid nanoparticles disclosed in patent applications U.S. 2006/0240554, U.S. 2008/0020058 and PCT/US08/002006.

### SUMMARY OF THE INVENTION

The instant invention provides for novel lipid nanoparticles and novel lipid nanoparticle components (specifically cationic lipids) that are useful for the delivery of nucleic acids, specifically siRNA, for therapeutic purposes.

### DETAILED DESCRIPTION OF THE INVENTION

The description below of the various aspects and embodiments of the invention is provided with reference to an exemplary gene ApoB (apolipoprotein B). The various aspects and embodiments of the invention are directed to and support the utility of novel lipid nanoparticles to deliver biologically active molecules, in particular, siRNA, to any target gene. (See US patent applications: US 2006/0240554 and US 2008/0020058).

The lipid nanoparticle components (cationic lipids) of the instant invention are useful components in a lipid nanoparticle for the delivery of nucleic acids, specifically siRNA.

One cationic lipid is:

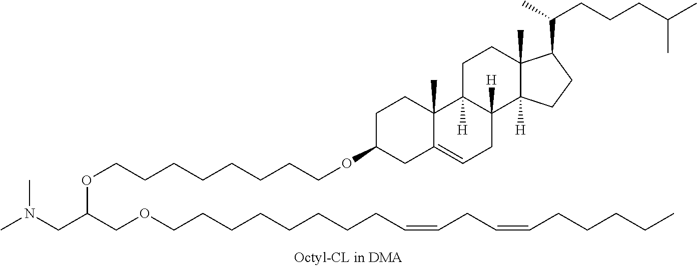

2-({8-[(3β)-cholest-5-en-3-yloxy]octyl}oxy)-N,N-dimethyl-3-[(9Z,12Z)-octadeca-9,12-dien-1-yloxy]propan-1-amine.

Another cationic lipid is:

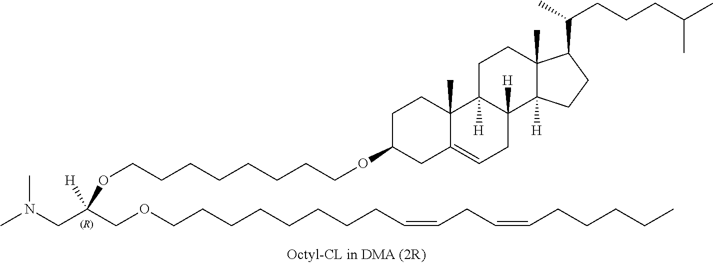

(2R)-2-({8-[(3β)-cholest-5-en-3-yloxy] octyl}oxy)-N,N-dimethyl-3-[(9Z,12Z)-octadeca-9,12-dien-1-yloxy]propan-1 -amine

Another cationic lipid is:

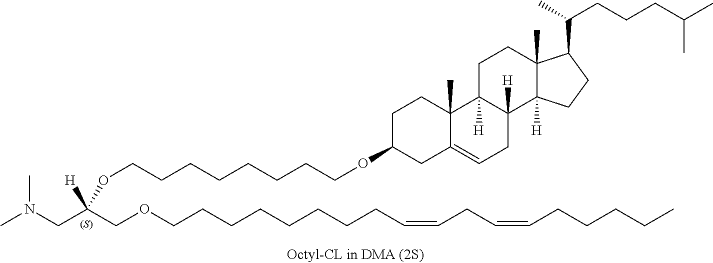

(25)-2-({8-[(3β)-cholest-5-en-3-yloxy]octyl}oxy)-N,N-dimethyl-3-[(9Z,12Z)-octadeca-9,12-dien-1-yloxy]propan-1-amine.

### LNP255 Compositions

The following lipid nanoparticle compositions of the instant invention are useful for the delivery of nucleic acids, specifically siRNA:

### Octyl-CLinDMA/Cholesterol/PEG-DMG 60/38/2;

### Octyl-CLinDMA (2R)/Cholesterol/PEG-DMG 60/38/2; and

### Octyl-CLinDMA (2S)/Cholesterol/PEG-DMG 60/38/2.

The following lipid nanoparticle compositions of the instant invention are useful for the delivery of nucleic acids, specifically siRNA:

### Octyl-CLinDMA/Cholesterol/PEG-DMG 58.9/39.4/1.6;

### Octyl-CLinDMA (2R)/Cholesterol/PEG-DMG 60.3/38.1/1.6; and

### Octyl-CLinDMA (2S)/Cholesterol/PEG-DMG 60.4/38.0/1.6.

In an embodiment, the invention features a lipid nanoparticle composition comprising one or more biologically active molecules (e.g., a polynucleotide such as a siRNA, siNA, antisense, aptamer, decoy, ribozyme, 2-5A, triplex forming oligonucleotide, or other nucleic acid molecule), cationic lipid selected from Octyl-CLinDMA, Octyl-CLinDMA (2R) and Octyl-CLinDMA (2S) or combinations thereof, neutral lipid which is (PEG-DMG), and cholesterol.

In another embodiment, the invention features a lipid nanoparticle composition comprising one or more siRNA molecules, cationic lipid selected from Octyl-CLinDMA, Octyl-CLinDMA (2R) and Octyl-CLinDMA (2S) or combinations thereof, neutral lipid which is (PEG-DMG), and cholesterol.

In another embodiment, the invention features a lipid nanoparticle composition comprising one or more siRNA molecules, Octyl-CLinDMA, PEG-DMG, and cholesterol.

In another embodiment, the invention features a lipid nanoparticle composition comprising one or more siRNA molecules, Octyl-CLinDMA (2R), PEG-DMG, and cholesterol.

In another embodiment, the invention features a lipid nanoparticle composition comprising one or more siRNA molecules, Octyl-CLinDMA (2S), PEG-DMG, and cholesterol.

In another embodiment, the invention features a lipid nanoparticle composition comprising siRNA molecules, cationic lipid selected from Octyl-CLinDMA, Octyl-CLinDMA (2R) and Octyl-CLinDMA (2S) or combinations thereof, neutral lipid which is (PEG-DMG), and cholesterol.

In another embodiment, the invention features a lipid nanoparticle composition comprising siRNA molecules, Octyl-CLinDMA, PEG-DMG, and cholesterol.

In another embodiment, the invention features a lipid nanoparticle composition comprising siRNA molecules, Octyl-CLinDMA (2R), PEG-DMG, and cholesterol.

In another embodiment, the invention features a lipid nanoparticle composition comprising siRNA molecules, Octyl-CLinDMA (2S), PEG-DMG, and cholesterol.

In another embodiment, the ratio of the lipids in the lipid nanoparticle composition has a mole percent range of 25-75 for the cationic lipid (Octyl-CLinDMA, Octyl-CLinDMA (2R) and Octyl-CLinDMA (2S)) with a target of 45-65, the cholesterol has a mole percent range from 30-50 with a target of 30-50 and the PEG-DMG lipid has a mole percent range from 1-6 with a target of 1-5.

In another embodiment, the ratio of the lipids in the lipid nanoparticle composition has a mole percent range of 40-65 for the cationic lipid (Octyl-CLinDMA, Octyl-CLinDMA (2R) and Octyl-CLinDMA (2S)) with a target of 50-60, the cholesterol has a mole percent range from 30-50 with a target of 38-48 and the PEG-DMG lipid has a mole percent range from 1-6 with a target of 1-5.

In another embodiment, the ratio of the lipids in the lipid nanoparticle composition has a mole percent range of 55-65 for the cationic lipid (Octyl-CLinDMA, Octyl-CLinDMA (2R) and Octyl-CLinDMA (2S)), the cholesterol has a mole percent range from 37-41 and the PEG-DMG lipid has a mole percent range from 1-3.

PEG-DMG is known in the art. (See US patent applications: US 2006/0240554 and US 2008/0020058).

Cholesterol is known in the art. (See US patent applications: US 2006/0240554 and US 2008/0020058).

In another embodiment, the invention features a method for delivering or administering a biologically active molecule (in particular, an siRNA) to a cell or cells in a subject or organism, comprising administering a formulated molecular composition of the invention under conditions suitable for delivery of the biologically active molecule component of the formulated molecular composition to the cell or cells of the subject or organism. In one embodiment, the formulated molecular composition is contacted with the cell or cells of the subject or organism as is generally known in the art, such as via parental administration (e.g., intravenous, intramuscular, subcutaneous administration) of the formulated molecular composition with or without excipients to facilitate the administration.

In another embodiment, the invention features a method for delivering or administering a biologically active molecule (in particular, an siRNA) to liver or liver cells (e.g., hepatocytes), kidney or kidney cells, tumor or tumor cells, CNS or CNS cells (e.g., brain, spinal cord), lung or lung cells, vascular or vascular cells, skin or skin cells (e.g., dermis or dermis cells, follicle or follicular cells), eye or ocular cells (e.g., macula, fovea, cornea, retina etc.), ear or cells of the ear (e.g., inner ear, middle ear, outer ear), in a subject or organism, comprising administering a foiinulated molecular composition of the invention under conditions suitable for delivery of the biologically active molecule component of the formulated molecular composition to the above described cells of the subject or organism. The formulated molecular composition is contacted with the above described cells of the subject or organism as is generally known in the art, such as via parental administration (e.g., intravenous, intramuscular, subcutaneous administration) or local administration (e.g., direct injection, direct dermal application, ionophoresis, intraocular injection, periocular injection, eye drops, implants, portal vein injection, pulmonary administration, catheterization, clamping, stenting etc.) of the formulated molecular composition with or without excipients to facilitate the administration.

In another embodiment, the invention features a formulated siRNA composition comprising short interfering ribonucleic acid (siRNA) molecules that down-regulate expression of a target gene or target genes. siRNA molecules (chemically modified or unmodified) are known in the art. (See US patent applications: US 2006/0240554 and US 2008/0020058).

In another embodiment, the invention features a formulated siRNA composition comprising a double stranded short interfering ribonucleic acid (siRNA) molecule that directs cleavage of a target RNA via RNA interference (RNAi), wherein the double stranded siRNA molecule comprises a first and a second strand, each strand of the siRNA molecule is about 18 to about 28 nucleotides in length or about 18 to about 23 nucleotides in length, the first strand of the siRNA comprises nucleotide sequence having sufficient complementarity to the target RNA for the siRNA molecule to direct cleavage of the target RNA via RNA interference, and the second strand of said siRNA molecule comprises nucleotide sequence that is complementary to the first strand.

In another embodiment, the invention features a formulated siRNA composition comprising a chemically synthesized double stranded short interfering ribonucleic acid (siRNA) molecule that directs cleavage of a target RNA via RNA interference (RNAi), wherein each strand of the siRNA molecule is about 18 to about 23 nucleotides in length; and one strand of the siRNA molecule comprises nucleotide sequence having sufficient complementarity to the target RNA for the siRNA molecule to direct cleavage of the target RNA via RNA interference.

In another embodiment, the invention features a formulated siRNA composition comprising a siRNA molecule that down-regulates expression of a target gene, for example, wherein the target gene comprises a target encoding sequence. In another embodiment, the invention features a siRNA molecule that down-regulates expression of a target gene, for example, wherein the target gene comprises a target non-coding sequence or regulatory elements involved in target gene expression.

An siRNA molecule may be used to inhibit the expression of target genes or a target gene family, wherein the genes or gene family sequences share sequence homology. Such homologous sequences can be identified as is known in the art, for example using sequence alignments. siRNA molecules can be designed to target such homologous sequences, for example using perfectly complementary sequences or by incorporating non-canonical base pairs, for example mismatches and/or wobble base pairs that can provide additional target sequences. In instances where mismatches are identified, non-canonical base pairs (for example, mismatches and/or wobble bases) can be used to generate siRNA molecules that target more than one gene sequence. In a non-limiting example, non-canonical base pairs such as UU and CC base pairs are used to generate siRNA molecules that are capable of targeting sequences for differing targets that share sequence homology. As such, one advantage of using siRNAs is that a single siRNA can be designed to include nucleic acid sequence that is complementary to the nucleotide sequence that is conserved between the homologous genes. In this approach, a single siRNA can be used to inhibit expression of more than one gene instead of using more than one siRNA molecule to target the different genes.

In another embodiment, the invention features a formulated siRNA composition comprising a siRNA molecule having RNAi activity against a target RNA, wherein the siRNA molecule comprises a sequence complementary to any RNA having target encoding sequence. Examples of siRNA molecules suitable for the formulations described herein are provided in International Application Serial Number US 04/106390 (WO 05/19453), which is hereby incorporated by reference in its entirety. Chemical modifications as described in PCT/US 2004/106390 (WO 05/19453), U.S. Ser. No. 10/444,853, filed May 23, 2003 U.S. Ser. No.

10/923,536 filed Aug. 20, 2004, U.S. Ser. No. 11/234,730, filed Sep. 23, 2005 or U.S. Ser. No. 11/299,254, filed Dec. 8, 2005, all incorporated by reference in their entireties herein.

An siRNA molecule may include a nucleotide sequence that can interact with a nucleotide sequence of a target gene and thereby mediate silencing of target gene expression, for example, wherein the siRNA mediates regulation of target gene expression by cellular processes that modulate the chromatin structure or methylation patterns of the target gene and prevent transcription of the target gene.

### EXAMPLES

Examples provided are intended to assist in a further understanding of the invention. Particular materials employed, species and conditions are intended to be further illustrative of the invention and not limitative of the reasonable scope thereof. The reagents utilized in synthesizing the cationic lipids are either commercially available or are readily prepared by one of ordinary skill in the art.

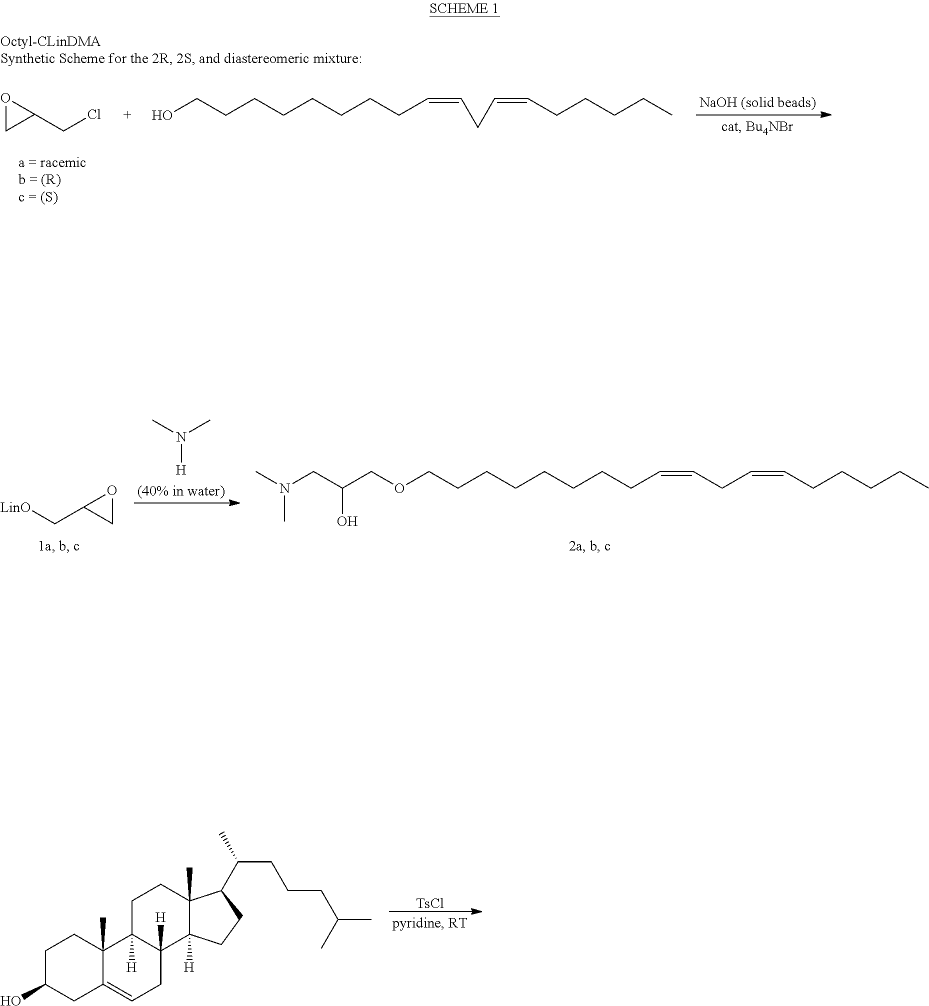

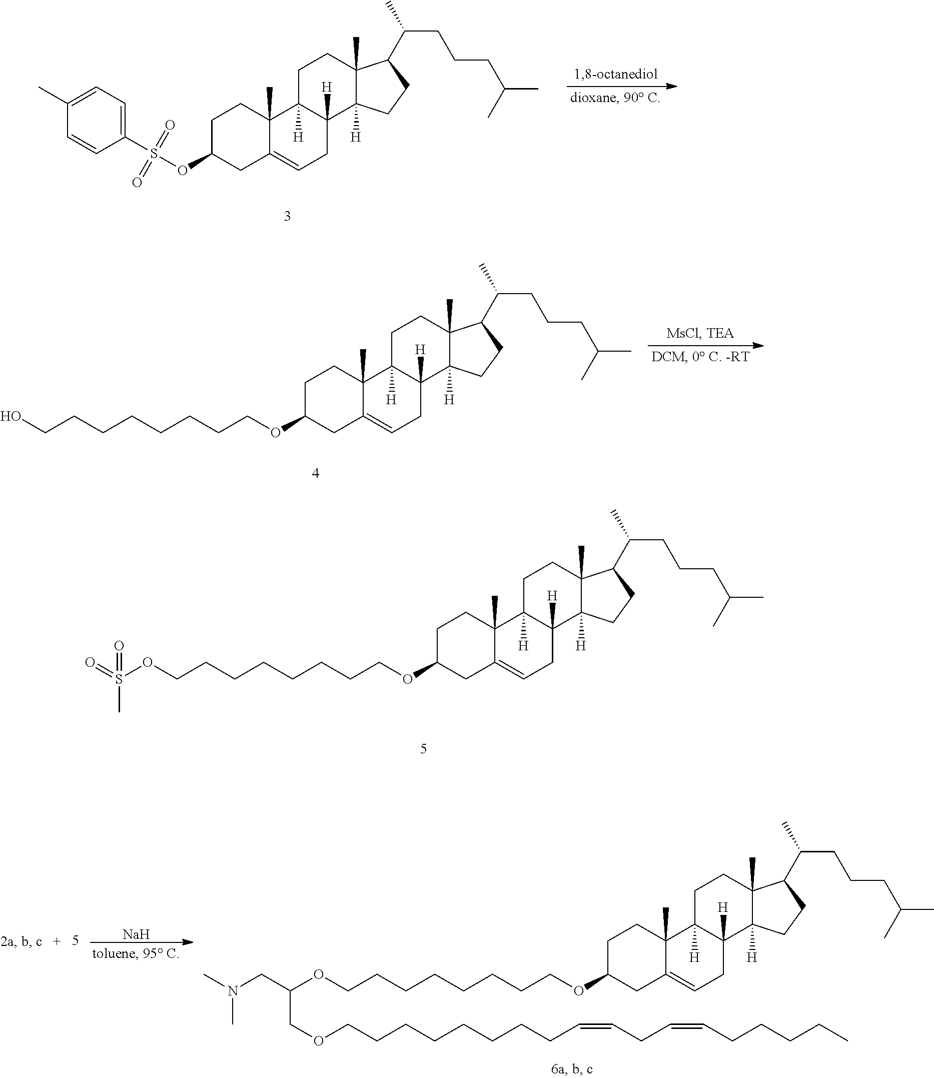

### Experimental Procedures:

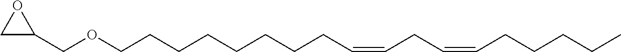

2-{[(9Z,12Z)-octadeca-9,12-dien-1-yloxy]methyl}oxirane (1a). Linoleyl alcohol (25 g, 94 mmol) and tetrabutylammonium bromide (1.51 g, 4.69 mmol) were weighed into a dry flask under nitrogen. Sodium hydroxide beads (5.63 g, 141 mmol) were added and the mixture was stirred for 5 minutes. Epichlorohydrin (13 g, 141 mmol) was added in a single portion, and the reaction was stirred overnight. The solution was diluted in ethyl acetate and filtered through a Buchner funnel to remove solids. Concentration in vacuo yielded the crude product as a colorless oil. The crude product was purified using normal phase chromatography, eluting with a gradient of 0-50% ethyl acetate in hexanes to afford 26.5 g (88%) of 2-{[(9Z,12Z)-octadeca-9,12-dien-1-yloxy]methyl}oxirane (1a) as a colorless oil. ¹H NMR (400 MHz, CDCl₃) δ5.40 (m, 4H), 3.70 (m, dd, J=11.2, J=2.8, 1H), 3.52-3.42 (m, 2H), 3.38 (m, 1H), 3.14 (m, 1H), 2.80-2.74 (m, 311), 2.6 (m, 1H), 2.10 (m, 4H), 1.60 (m, 2H), 1.40-1.22 (m, 16H), 0.88 (m, 3H).

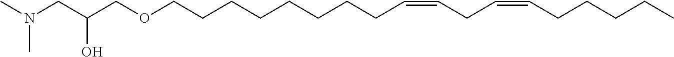

1-(dimethylarnino)-3-[(9Z,12Z)-octadeca-9,12-dien-1-yloxy]propan-2-ol (2a). 1a(37 g, 115 mmol) was dissolved in ethanol (1000 ml) in a high-pressure flask and cooled to 0° C. in an ice bath. Dimethylamine was bubbled into the solution. The flask was sealed and allowed to warm to 23° C. over 72 hours. The flask was vented, and nitrogen was bubbled through the solution for 30 minutes. The solution was concentrated in vacua to yield a pale yellow oil. The crude product was filtered through a pad of silica, and eluted with chloroform saturated with ammonia. The solvent was removed in vacuo to yield 1-(dimethylarnino)-3-[(9Z,12Z)-octadeca-9,12-dien-1 -yloxy]propan-2-ol (2a) (41.57 g, 99%) as a colorless oil. ¹H NMR (400 MHz, CDCl₃) δ5.44-5.28 (m, 411), 3.84 (m, 1H), 3.5-3.38 (m, 5H), 3.30 (s, 1H), 2.77 (t, J=6.4 Hz, 2H), 2.44-2.39 (m, 1H), 2.30-2.21 (m, 7H), 2.05 (m, 4H), 1.60 (m, 2H), 1.40-1.26 (m, 16H), 0.88 (t, J=7.2, 3H).

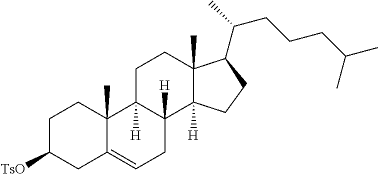

(3β)-cholest-5-en-3-yl 4-methylbenzenesulfonate (3). To a solution of cholesterol (100 g, 259 mmol) in pyridine (1500 mL) was added tosyl chloride (74 g, 388 mmol). The reaction was stirred for 16 hours. The solvent was removed in vacuo. The residue was dissolved in ethyl acetate and filtered through a pad of celite. The solvent was removed in vacua to yield the crude product as a residue. The residue was taken up in a small amount of DCM. Addition of methanol yielded a colorless precipitate. The product was collected by filtration through a Buchner funnel followed by rinses of cold methanol to give 122 g (87%) of (3β)-cholest-5-en-3-yl 4-methylbenzenesulfonate (3) as colorless crystals. ¹H NMR (400 MHz, CDCl₃)δ7.79 (d, J=8.0 Hz, 2H), 7.32 (d, J=8 Hz, 2H), 5.30 (m, 1H), 4.32 (m, 1H), 2.45 (m, 4H), 2.25 (m, 1H), 2.05-1.90 (m, 211), 1.85-1.65 (m, 4H), 1.58-1.25 (m, 12H), 1.12-1.05 (in, 5H), 1.04- 0.94 (m, 10H), 0.66 (s, 3H).

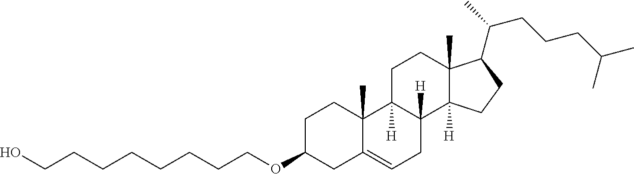

8-[(3β-cholest-5-en-3-yloxy]octan-1-ol (4). 1,8-Octanediol (32.4 g, 222 mmol) was dissolved in 100 mL dioxane and heated to 90° C. until dissolution of solids was complete. To this solution was added a solution of 3 (6 g, 11.1 mmol) dissolved in 20 mL dioxane through an addition funnel. After 16 hours, the reaction was cooled and concentrated in vacuo. The residue was diluted in DCM and filtered through a Buchner funnel to remove precipitate. The resulting solution was concentrated in vacuo to yield the crude product as a viscous oil. Purified using silica gel chromatography and a gradient of 0- 100% ethyl acetate in hexanes to yield pure 8-[(3β)-cholest-5-en-3-yloxy]octan-1-ol (4) (5.2 g, 91%) as a colorless solid. ¹H NMR (400 MHz, CDC₁₃)δ6 5.35 (m, 111), 3.64 (q, J=6.4 Hz, 2H) 3.44 (t, J=6.4 Hz, 2H), 3.12 (m, 1H), 2.35 (m, 1H), 2.20 (m, 1H), 2.03-1.79 (m, 5H), 1.59-1.40 (m, 14H), 1.33 (br s, 13H), 1.22-1.05 (m, 10H), 1.00 (s, 4H), 0.93-0.83 (m, 10H), 0.65 (s, 3H).

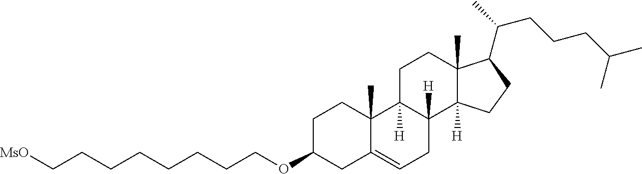

8-[(3β)-cholest-5-en-3-yloxy]octyl methanesulfonate (5). To a cooled (0° C.) solution of 4 (3.68 g, 7.15 mmol) and triethylamine (1.49 mL, 8.58 mmol) in 80 mL of DCM was added methanesulfonylchloride (0.69 mL, 8.93 mmol) dropwise over 15 minutes. The solution stirred for 15 minutes at 0° C., and then was allowed to warm to 23° C. over 1.5 hours. The reaction was quenched with brine and extracted with DCM (2×). The organic layers were combined, dried over sodium sulfate, and concentrated in vacuo to yield 4.25 g (100%) of the crude 8-[(3β)-cholest-5-en-3-yloxy]octyl methanesulfonate (5) as a colorless semi-solid. ¹H NMR (400 MHz, CDC₁₃)δ5.34 (m, 1H), 4.22 (t, J=6.8 Hz, 2H) 3.65 (m, 2H) 3.44 (t, J=6.4 Hz, 2H), 3.09 (m, 2H), 3.00 (s, 3H), 2.35 (m, 1H), 2.20 (m, 1H), 2.04-1.80 (m, 5H), 1.74 (m, 2H), 1.68 (s, 2H), 1.60-1.24 (m, 30H), 1.22-1.05 (m, 10H), 1.00 (s, 4H), 0.93- 0.83 (m, 10H), 0.65 (s, 3H).

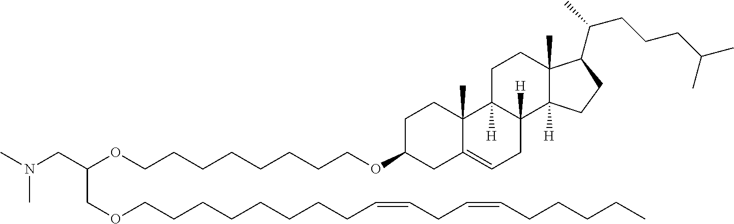

2-({8-[(3β)-cholest-5-en-3-yloxy]octyl}oxy)-N,N-dimethyl-3-[(9Z,12Z)-ftetadeca-9,12-dien-1-yloxy]propan-1-amine (6a). To a solution of 2a (5 g, 13.6 mmol) in 80 mL toluene was added 60% sodium hydride dispersion in mineral oil (1.1 g, 27.2 mmol). The solution was heated to 95° C. and then a solution of 5 (9.68 g, 16.3 mmol) in 20 mL toluene was added dropwise over 1 hour. After an additional 1.5 hours, the solution was cooled and quenched with drops of methanol. Brine (100 mL) was added, and the solution was extracted with ethyl acetate (2×.) Organics were combined and filtered through a short pad of celite, rinsing with ethyl acetate. The solution was dried over sodium sulfate and concentrated in vacua to yield the crude product as a yellow oil. Silica gel chromatography with a gradient of 0-100% ethyl acetate in hexanes afforded 7.4 g (63%) of 2-({8-[(3β)-cholest-5-en-3-yloxy]octyl}oxy)-N,N-dimethyl-3-[(9Z,12Z)-octadeca-9,12-dien-1-yloxy]propan-1-amine (6₄) as pale yellow oil. ¹H NMR (400 MHz, CDCl₃)δ5.34 (in, 4H), 3.61-3.42 (m, 10H), 3.12 (m, 1H), 2.77 (t, J=6.4 Hz, 2H), 2.40 (m, 3H), 2.28 (br s, 6H), 2.20 (m, 1H), 2.05 (m, 611), 1.85 (m, 3H), 1.61-1.46 (m, 14H), 1.40-1.22 (m, 30H), 1.15 (m, 8H), 1.0 (m, 5H), 0.90 (m, 14H), 0.68 (s, 3H). ESI HRMS m/z calculated for C58H105NO3 [M+1] 864.8172, found 864.8147.

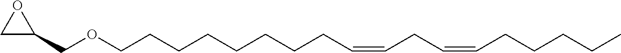

(2R)-2-{[(9Z,12Z)-Octadeca-9,12-digin-1-yloxy]methyl}oxirane (1b). Linoleyl alcohol (48 g, 180 mmol), sodium hydroxide (7.21 g, 180 mmol) and tetrabutylammonium bromide (2.90 g, 9.01 mmol) were combined in a 200 mL flask, stirred for 10 min, and then (R)-(-)-epichlorohydrin (21.19 ml, 270 mmol) was added. After 5 hours, 50% more of the chloride, hydroxide and salt were added and stirred overnight, then diluted with 1500 mL EtOAc and extracted with water, brine, dry (Na₂SO₄), and filtered. Solvent was removed in vacuo, and hivac distilled through a 6″ Vigreux column (mantle temp 300° C., head temp 145-155° C.) to get 45.1 g (0.140 mol, 78%) of (2R)-2-{[(9Z,12Z)-octadeca-9,12-dien-1-yloxy]methyl}oxirane (1b) as a water white oil. ¹H NMR (400 MHz, CDCl₃)δ5.40 (m, 4H), 3.70 (m, dd, J=11.2, J=2.8, 1H), 3.52-3.42 (m, 2H), 3.38 (m, 1H), 3.14 (m, 1H), 2.80-2.74 (m, 3H), 2.6 (m, 1H), 2.10 (m, 4H), 1.60 (m, 2H), 1.40-1.22 (m, 16H), 0.88 (m, 3H).

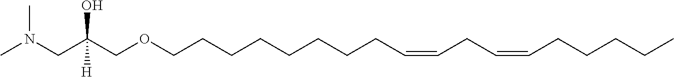

(2R)-1-(Dimethylamillo)-3-[(9Z,12Z)-octadeca-9,12-dien-1-yloxy]propan-2-ol (2b). 1b (10 g, 31.0 mmol) was dissolved in 200 mL of a 5.6 M (33%) dimethylamine solution in ethanol and stirred overnight. The solvent was removed in vacuo to get 11.21 g (30.5 mmol, 98%) of (2R)-1-(dimethylamino)-3-[(9Z,12Z)-octadeca-9,12-dien-1-yloxy]propan-2-ol (2b) which was used without further purification. ¹H NMR (400 MHz, CDCI₃)δ5.44-5.28 (m, 4H), 3.84 (m, 1H), 3.5-3.38 (m, 5H), 3.30 (s, 1H), 2.77 (t, J=6.4 Hz, 2H), 2.44-2.39 (m, 1H), 2.30-2.21 (m, 7H), 2.05 (m, 4H), 1.60 (m, 2H), 1.40-1.26 (m, 16H), 0.88 (t, J=7.2, 3H).

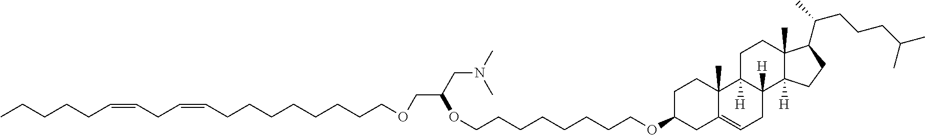

(2R)-2-({8-[(3β)-Cholest-5-en-3-yloxy]octyl}oxy)-N,N-dimethyl-3[(9Z,12Z)-octadeca-9,12-dien-1-yloxy]propan-1-amine (6b). 2b was placed in toluene (100 ml) under a nitrogen atmosphere and sodium hydride (0.479 g, 11.97 mmol) was slowly added, then heated to 80-90° C., then 5 (4.26 g, 7.18 mmol) was added in toluene (5 ml) dropwise over a 6 hr. period, heated overnight, and cooled to 0° C. 50 mL EtOH was slowly added, stirred 30 min and then the solvent was removed. 300 mL EtOAc was added and filtered through a celite pad. Solvent was removed, then passed through a 8″×4.5″ silica pad, eluted with 3:1 H/EtOAc to 100% EtOAc to yield 4.2 g (4.86 mmol, 81%) (2R)-2-({8-[(3β)-cholest-5-en-3-yloxy]octyl}oxy)-N,N-dimethyl-3-[(9Z,12Z)-octadeca-9,12-dien-1-yloxy]propan-1-amine (6b). ¹H NMR (400 MHz, CDCl₃)δ5.34 (m, 411), 3.61-3.42 (m, 10H), 3.12 (m, 1H), 2.77 (t, J=6.4 Hz, 2H), 2.40 (m, 3H), 2.28 (br s, 6H), 2.20 (m, 1H), 2.05 (m, 6H), 1.85 (in, 3H), 1.61-1.46 (m, 14H), 1.40-1.22 (m, 30H), 1.15 (m, 8H), 1.0 (in, 5H), 0.90 (m, 14H), 0.68 (s, 3H). ESI HRMS m/z calcd for C58H105NO3 [M+1] 864.8094, found 864.8167

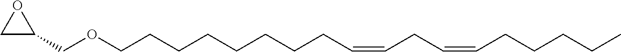

(2S)-2-{[(9Z,12Z)-Octadeca-9,12-dien-1-yloxy]metbyl}oxirane (1c). In a similar manner to the above example, linoleyl alcohol (50 g, 188 mmol), sodium hydroxide (7.51 g, 188 mmol), tetrabutylammonium bromide (3.02 g, 9.38 mmol) and (S)-(+)-epichlorohydrin (22.01 ml, 281 mmol) were reacted to get 47.4 (0.148 mol, 79%) of (2S)-2-{[(9Z,12Z)-octadeca-9,12-dien-1-yloxy]methyl}oxirane (1c) as a water white oil after distillation (mantle temp 293-7° C., head temp 150-155° C.). ¹H NMR (400 MHz, CDCl₃)δ5.40 (m, 4H), 3.70 (m, dd, J=11.2, J=2.8, 1H), 3.52-3.42 (m, 2H), 3.38 (m, 1H), 3.14 (m, 1H), 2.80-2.74 (m, 3H), 2.6 (m, 1H), 2.10 (m, 4H), 1.60 (m, 2H), 1.40-1.22 (m, 16H), 0.88 (m, 3H).

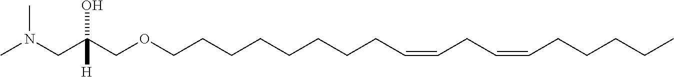

(2S)-1-(Dimethylamino)-3-[(9Z,12Z)-octadeca-9,12-dien-1-yloxy]propan-2-ol (2c). In a similar manner as the above example, 5.1 g (15.81 mmol) of 1c was reacted in 100 mL of a 5.6 M (33%) dimethylamine solution in ethanol to give 5.8 g (15.78 mmol, 100%) of (2S)-1-(dimethylamino)-3-[(9Z,12Z)-octadeca-9,12-dien-1-yloxy]propan-2-ol (2c). ¹H NMR (400 MHz, CDC₁₃)δ5.44-5.28 (m, 4H), 3.84 (m, 1H), 3.5-3.38 (m, 5H), 3.30 (s, 1H), 2.77 (t, J=6.4 Hz, 2H), 2.44-2.39 (m, 1H), 2.30-2.21 (m, 7H), 2.05 (m, 4H), 1.60 (m, 2H), 1.40-1.26 (m, 16H), 0.88 (t, J=7.2, 3H).

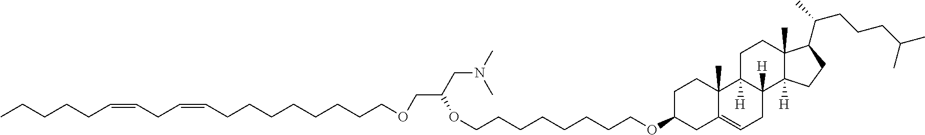

(2S)-2-({8-[(3β)-Cholest-5-en-3-yloxy]octyl}oxy)-N,N-dimethyl-3-[(9Z,12Z)-octadeca-9,12-dien-1-yloxy]propan-1-amine (6c). In a similar manner as the above example, 2.2 g (5.98 mmol) of 2c was reacted with sodium hydride (0.479 g, 11.97 mmol) and 5 (4,26 g, 7.18 mmol) to give 4.1 g (4.74 mmol, 79%) of (2S)-2-({8-[(3β)-cholest-5-en-3-yloxy]octyl}oxy)-N,N-dimethyl-3-[(9Z,12Z)-octadeca-9,12-dien-1-yloxy]propan-1-amine (6c). ¹H NMR (400 MHz, CDCl₃)δ5.34 (m, 4H), 3.61-3.42 (m, 10H), 3.12 (m, 1H), 2.77 (t, J=6.4 Hz, 2H), 2.40 (m, 3H), 2.28 (br s, 6H), 2.20 (m, 1H), 2.05 (m, 6H), 1.85 (m, 3H), 1.61-1.46 (m, 14H), 1.40-1.22 (m, 30H), 1.15 (m, 8H), 1.0 (m, 5H), 0.90 (m, 14H), 0.68 (s, 3H). ESI HRMS m/z calcd for C58H105NO3 [M+1] 864.8094, found 864.8177

### Scheme 4

### LNP255 Compositions

### LNP255 Process Description:

The Lipid Nano-Particles (LNP) are prepared by an impinging jet process. The particles are formed by mixing equal volumes of lipids dissolved in alcohol with siRNA dissolved in a citrate buffer. The lipid solution contains a cationic (Octyl-CLinDMA, Octyl-CLinDMA (2R) and Octyl-CLinDMA (2S)), helper (cholesterol) and PEG (PEG-DMG) lipids at a concentration of 8-12 mg/mL with a target of 10 mg/mL in an alcohol (for example ethanol). The ratio of the lipids has a mole percent range of 25-75 for the cationic lipid with a target of 45-65, the helper lipid has a mole percent range from 25-75 with a target of 30-50 and the PEG lipid has a mole percent range from 1-6 with a target of 2-5. The siRNA solution contains one or more siRNA sequences at a concentration range from 0.7 to 1.0 mg/mL with a target of 0.8 -0.9 mg/mL in a sodium citrate: sodium chloride buffer pH 4. The two liquids are mixed in an impinging jet mixer instantly forming the LNP. The tubing ID has a range from 0.25 to 1.0 mm and a total flow rate from 10-120 mL/min. The combination of flow rate and tubing ID has effect of controlling the particle size of the LNPs between 50 and 200 nm. The mixed LNPs are held from 30 minutes to 48 hrs prior to a dilution step. The dilution step comprises similar impinging jet mixing which instantly dilutes the LNP. This process uses tubing IDs ranging from 1 mm ID to 5 mm ID and a flow rate from 40 to 360 mL/min. The LNPs are concentrated and diafiltered via an ultrafiltration process where the alcohol is removed and the citrate buffer is exchanged for the final buffer solution such as phosphate buffered saline. The ultrafiltration process uses a tangential flow filtration format (TFF). This process uses a membrane nominal molecular weight cutoff range from 30-100 KD. The membrane format can be hollow fiber or flat sheet cassette. The TFF processes with the proper molecular weight cutoff retains the LNP in the retentate and the filtrate or permeate contains the alcohol; citrate buffer; final buffer wastes. The TFF process is a multiple step process with an initial concentration to a siRNA concentration of 1-3 mg/mL. Following concentration, the LNPs solution is diafiltered against the final buffer for 15-20 volumes to remove the alcohol and exchange the buffers. The final steps of the LNP process are to sterile filter the LNP and vial the product.

### Analytical Procedure:

1) siRNA Concentration

The siRNA duplex concentrations are determined by Strong Anion-Exchange High-Performance Liquid Chromatography (SAX-HPLC) using Waters 2695 Alliance system (Water Corporation, Milford Mass.) with a 2996 PDA detector. The LNPs, otherwise refered to as RNAi Delivery Vehicles (RDVs), are treated with 0.5% Triton X-100 to free total siRNA and analyzed by SAX separation using a Dionex BioLC DNAPac PA 200 (4×250 mm) column with UV detection at 254 nm. Mobile phase is composed of A: 25 mM NaClO₄, 10 mM Tris, 20% EtOH, pH 7.0 and B: 250 mM NaClO₄, 10 mM Tris, 20% EtOH, pH 7.0 with liner gradient from 0-15 mM and flow rate of 1 ml/min. The siRNA amount is determined by comparing to the siRNA standard curve.

### 2) Encapsulation Rate

Fluorescence reagent SYBR Gold is employed for RNA quantitation to monitor the encapsulation rate of RDVs. RDVs with or without Triton X-100 are used to determine the free siRNA and total siRNA amount. The assay is performed using a SpectraMax M5e microplate spectrophotometer from Molecular Devices (Sunnyvale, Calif.). Samples are excited at 485 rim and fluorescence emission was measured at 530 nm. The siRNA amount is determined by comparing to the siRNA standard curve.

Encapsulation rate=(1−free siRNA/total siRNA)×100%

### 3) Particle Size and Polydispersity

RDVs containing 1 μg siRNA are diluted to a final volume of 3 ml with 1×PBS. The particle size and polydispersity of the samples is measured by a dynamic light scattering method using ZetaPALS instrument (Brookhaven Instruments Corporation, Holtsville, N.Y.). The scattered intensity is measured with He-Ne laser at 25° C. with a scattering angle of 90° .

### 4) Zeta Potential Analysis

RDVs containing 1 siRNA are diluted to a final volume of 2 ml with milliQ H₂O. Electrophoretic mobility of samples is determined using ZetaPALS instrument (Brookhaven Instruments Corporation, Holtsville, N.Y.) with electrode and He—Ne laser as a light source. The Smoluchowski limit is assumed in the calculation of zeta potentials.

### 5) Lipid Analysis

Individual lipid concentrations are determined by Reverse Phase High-Performance Liquid Chromatography (RP-HPLC) using Waters 2695 Alliance system (Water Corporation, Milford Mass.) with a Corona charged aerosol detector (CAD) (ESA Biosciences, Inc, Chelmsford, Mass.). Individual lipids in RDVs are analyzed using a Agilent Zorbax SB-C18 (50×4.6 mm, 1.8 μm particle size) column with CAD at 60° C. The mobile phase is composed of A: 0.1% TFA in H₂O and B: 0.1% TFA in IPA. The gradient is 75% mobile phase A and 25% mobile phase B from time 0 to 0.10 min; 25% mobile phase A and 75% mobile phase B from 0.10 to 1.10 min; 25% mobile phase A and 75% mobile phase B from 1.10 to 5.60 min; 5% mobile phase A and 95% mobile phase B from 5.60 to 8.01 min; and 75% mobile phase A and 25% mobile phase B from 8.01 to 13 min with flow rate of 1 ml/min. The individual lipid concentration is determined by comparing to the standard curve with all the lipid components in the RDVs with a quadratic curve fit. The molar percentage of each lipid is calculated based on its molecular weight.

Utilizing the above described LNP process, specific LNPs with the following ratios were identified:

### Nominal Composition:

### Octyl-CLinDMA/Cholesterol/PEG-DMG 60/38/2;

### Octyl-CLinDMA (2R)/Cholesterol/PEG-DMG 60/38/2; and

### Octyl-CLinDMA (2S) / Cholesterol/PEG-DMG 60/38/2.

Final composition:

### Octyl-CLinDMA/Cholesterol/PEG-DMG 58.9/39.4/1.6;

### Octyl-CLinDMA (2R)/Cholesterol/PEG-DMG 60.3/38.1/1.6; and

### Octyl-CLinDMA (25)/Cholesterol/PEG-DMG 60.4/38.0/1.6.

### Physical Characterization of ApoB LNPs

| | | Composition | | |
|---|---|---|---|---|
| siRNA | LNP255 | Cationic lipid | cholesterol | PEG-DMG |
| ApoB | Octyl-CLinDMA | 58.9 | 39.4 | 1.6 |
| | (R&S) | | | |
| ApoB | Octyl-CLinDMA | 60.3 | 38.1 | 1.6 |
| | (2R) | | | |
| ApoB | Octyl-CLinDMA | 60.4 | 38.0 | 1.6 |
| | (2S) | | | |

| | ApoB siRNA | Encapsu- | Particle | | Zeta |
|---|---|---|---|---|---|
| | Concentration | lation | Size | Poly- | Potential |
| LNP | (mg/mL) | rate (%) | (nm) | dispersity | (mV) |
| LNP255 | 2.77 | 93 | 125.8 | 0.08 | 2.9 |
| (R&S) | | | | | |
| LNP255 | 2.68 | 93.5 | 121.9 | 0.05 | 3.4 |
| (2R) | | | | | |
| LNP255 | 2.83 | 92.8 | 125.5 | 0.06 | 3.8 |
| (2S) | | | | | |

### Example 1

### In Vivo Evaluation of Efficacy:

LNP255 (R/S) 58.9/39.4/1.6 and the diastereomer specific LNP255(2R) 60.3/38.1/1.6 and LNP255(2S) 60.4/38.0/1.6 nanoparticles were evaluated for in vivo efficacy in mice. The siRNA employed targets the mouse mRNA transcript (nm009693) coding for the gene ApoB (apolipoprotein B).

| ApoB siRNA | |
|---|---|
| 5′-iB-CUUUAACAAUUCCUGAAAUTT-iB 3′ | (SEQ. ID. 1) |
| 3′-UUGAAAUUGUUAAGGACUUUA-5′ | (SEQ. ID. 2) |
| AUGC-Ribose | |
| iB-Inverted deoxy abasic | |
| UC-2′ Fluoro | |
| AGT-2′ Deoxy | |
| AGU-2′ OCH₃ | |

Mice were tail vein injected with the siRNA containing nanoparticles at doses of 0.3, 1, 3 and 9 mg/kg (dose based on siRNA content) in a volume of 0.2 mL, PBS vehicle. Three hours post dose, mice were bled retro-orbitally to obtain plasma for cytokine analysis. Twenty-four hours post dose, mice were sacrificed and liver tissue samples were immediately preserved in RNALater (Ambion). Preserved liver tissue was homogenized and total RNA isolated using a Qiagen bead mill and the Qiagen miRNA-Easy RNA isolation kit following the manufacturer's instructions. Liver ApoB mRNA levels were determined by quantitative RT-PCR. Message was amplified from purified RNA using a commercial probe set (Applied Biosystems Cat. No.

Mm01545156_m1). The PCR reaction was run on an ABI 7500 instrument with a 96-we11 Fast Block. The ApoB mRNA level is normalized to the housekeeping PP1B (NM 011149) mRNA. PPIB mRNA levels were determined by RT-PCR using a commercial probe set (Applied Biosytems Cat. No. Mm00478295_m1). Results are expressed as a ratio of ApoB mRNA/PPIB mRNA. All mRNA data is expressed relative to the PBS control dose.

Mouse In Vivo Efficacy Data:

Decreases in Apo mRNA levels, relative to the PBS control, were observed for all three LNP compositions in a dose dependent manner. Differences in mRNA levels, versus the PBS control, were significant at a CI of>99% for all LNP compositions at all dose levels. There were no statistically significant differences in mRNA knockdown efficacy between the different LNP compositions at a given dose level.

What is claimed is:

1. A cationic lipid which is selected from:
2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA);
(2R)-2-({8- [(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)); and
(2S)-2-({8-[(3β)-cholest-5-en-3-yloxy]octyl}oxy)-N,N-dimethyl-3- [(9Z,12Z)-octadeca-9,12-dien-1-yloxy]propan-1-amine (Octyl-CLinDMA (2S)).

2. A lipid nanoparticle composition comprising one or more biologically active molecules, cationic lipid selected from Octyl-CLinDMA, Octyl-CLinDMA (2R) and Octyl-CLinDMA (2S) or combinations thereof, neutral lipid which is (PEG-DMG), and cholesterol.

3. A lipid nanoparticle composition comprising one or more siRNA molecules, cationic lipid selected from Octyl-CLinDMA, Octyl-CLinDMA (2R) and Octyl-CLinDMA (2S) or combinations thereof, neutral lipid which is (PEG-DMG), and cholesterol.

4. A lipid nanoparticle composition of claim 3 comprising siRNA molecules, Octyl-CLinDMA, PEG-DMG, and cholesterol.

5. A lipid nanoparticle composition of claim 3 comprising siRNA molecules, Octyl-CLinDMA (2R), PEG-DMG, and cholesterol.

6. A lipid nanoparticle composition of claim 3 comprising siRNA molecules, Octyl-CLinDMA (2S), PEG-DMG, and cholesterol.

7. A lipid nanoparticle composition of claim 3, wherein said Octyl-CLinDMA, PEG-DMG, and cholesterol have a molar ratio of 60/38/2.

8. A lipid nanoparticle composition of claim 3, wherein said Octyl-CLinDMA (2R), PEG-DMG, and cholesterol have a molar ratio of 60/38/2.

9. A lipid nanoparticle composition of claim 3, wherein said Octyl-CLinDMA (2S), PEG-DMG, and cholesterol have a molar ratio of 60/38/2.


SEQUENCE LISTING (2 sequences)

SEQ ID NO: 1 - DNA, 21 bp, Artificial Sequence
  Enzymatic Nucleic Acid
cuuuaacaauuccugaaautt

SEQ ID NO: 2 - RNA, 21 nt, Artificial Sequence
  Enzymatic Nucleic Acid
uugaaauuguuaaggacuuua